(19) United States
(12) Patent Application Publication
(10) Pub. No.: US 2016/0051556 A1
(43) Pub. Date: Feb. 25, 2016

(54) Method and Pharmaceutical Composition for use in the Treatment of Chronic Liver Diseases Associated with a Low Hepcidin Expression

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR); CENTRE NATIONAL DE LA RECHERCHE SCIENTIFIQUE (CNRS), Paris (FR)

(72) Inventors: Marie-Paule Roth, Toulouse (FR); Helene Coppin, Toulouse (FR)

(21) Appl. No.: 14/392,086

(22) PCT Filed: Mar. 21, 2014
(86) PCT No.: PCT/EP2014/055754

§ 371 Date: Sep. 21, 2015

(30) Foreign Application Priority Data
Mar. 21, 2013 (EP) 13305345.4

(51) Int. Cl. A61K 31/5377; A61K 38/22

(52) U.S. Cl. CPC A61K 31/5377; A61K 38/22

(57) ABSTRACT

The present invention relates to an isolated EGF receptor agonist for use in the treatment of chronic liver diseases associated with a low hepcidin expression such as alcoholic liver disease, chronic hepatitis C, or genetic hemochromatosis.

### RELATED APPLICATION

The present application claims priority to European Patent Application No. EP 13305345.4, which was filed on Mar. 21, 2013. The European patent application is incorporated herein by reference in its entirety.

### FIELD OF THE INVENTION

The present invention relates to a method of treating chronic liver diseases with an Epidemial Growth Factor receptor (EGFR) antagonist. More specifically, it concerns use of an EGFR antagonist, for the treatment of chronic liver diseases associated with a low hepcidin expression such as alcoholic liver disease, chronic hepatitis C, or genetic hemochromatosis.

### BACKGROUND OF THE INVENTION

Iron is an important cofactor for essential cell functions such as oxygen transport, energy metabolism, and DNA synthesis. However, iron may also be dangerous as a catalyst of free radical reactions. Each day, the adult human body requires approximately 25 mg of iron for hemoglobin synthesis. The majority of this iron is supplied by macrophages which recycle iron from senescent erythrocytes. Only 1-2 mg is obtained through the absorption of dietary iron by duodenal enterocytes. Excess iron that is not consumed in erythropoiesis or other cellular processes is stored primarily in the liver and in reticuloendothelial macrophages. Whereas the body may modulate the absorption of dietary iron, no regulated mechanism for iron excretion from the body has been identified. Therefore, to ensure sufficient availability of iron for hemoglobin synthesis and other metabolic processes while avoiding the oxidative damage to cells that may result from excess free iron, iron balance must be tightly regulated (1,2).

Hepcidin, a circulating hormone produced primarily by the liver, plays a central role in the regulation of systemic iron homeostasis (3). Hepcidin binds to ferroportin, the only known iron export channel from cells into the plasma, highly expressed at the basolateral membrane of enterocytes and the plasma membrane of macrophages. Hepcidin binding leads to the internalization and degradation of ferroportin in lysosomes, thus decreasing the absorption of dietary iron and the release of recycled iron from macrophages (4). The essential role of hepcidin in the maintenance of systemic iron balance has been demonstrated in mouse models. Mice lacking hepcidin expression develop systemic iron overload (5), whereas transgenic mice overexpressing hepcidin exhibit severe iron deficiency anemia (6). In humans, loss-of-function mutations in the hepcidin gene HAMP cause juvenile hemochromatosis, an autosomal recessive disorder characterized by severe iron deposition in multiple organs, including the liver, heart, and endocrine tissues (7).

Recent advances have been made in the understanding of the molecular mechanisms through which hepcidin expression is modulated to influence systemic iron balance. Iron overload induces the expression of bone morphogenetic protein BMP6, a member of the TGF-β superfamily of ligands(8). Binding of BMP6 to paired serine/threonine kinase receptors results in phosphorylation of receptor-associated SMAD1/5/8 proteins, which after complexing with the common mediator protein SMAD4, translocate to the nucleus and modulate hepcidin gene transcription by binding to specific sequences in its promoter. Hemojuvelin (HJV) functions as an essential coreceptor for BMP6. Mice with disruption of either Bmp6 (9,10) or the hemojuvelin gene (11,12) exhibit hepcidin deficiency and severe iron overload, confirming the central role of these two molecules in the hepatic BMP signaling pathway that promotes hepcidin expression. Interestingly however, there are considerable, and still unexplained, gender differences in residual hepcidin expression and in the severity of tissue iron loading in both hemojuvelin- and Bmp6-deficient mice.

Clinical data have shown that men and women exhibit significant disparities in the progression of liver diseases such as alcoholic liver disease, chronic hepatitis C, or genetic hemochromatosis, all of which have been reported associated with altered hepcidin expression. These disparities have been attributed at least in part to gender-related variations in the regulation of iron metabolism (14) and it was hypothesized that further understanding of their underlying mechanisms may lead to the development of novel treatment strategies for chronic liver diseases associated with elevated hepcidin expression.

Accordingly, there is a need for a new therapeutic strategy of chronic liver diseases that restore hepcidine expression in the liver, particularly in male subject.

### SUMMARY OF THE INVENTION

By using murine models, the inventors took advantage of the very significant differences in hepcidin expression and iron stores observed between Bmp6-deficient males and females to explore the role of sexual hormones in the regulation of iron metabolism.

The inventors show that testosterone robustly represses hepcidin transcription by upregulating EGFR signaling and that selective EGFR inhibition in males markedly increases hepcidin expression. In males where the effects of testosterone and Bmp6-deficiency on hepcidin downregulation are combined, hepcidin is more strongly repressed than in females and iron accumulates massively not only in the liver but also in the pancreas, heart and kidneys. Accordingly, the inventors show that blocking EGFR constitutes an alternative therapeutic axis in chronic liver diseases and allows restoring hepcidin expression in the liver.

The present invention therefore provides antagonists of the EGF receptor (EGFR), for a novel use in the treatment of chronic liver diseases, more particularly in a male subject.

### DETAILED DESCRIPTION OF THE INVENTION

In a first aspect the invention provides an antagonist of the EGF receptor (EGFR), for use in treating chronic liver diseases associated with a low hepcidin expression.

In a second aspect the invention provides an inhibitor of EGFR, or EGF expression for use in treating chronic liver diseases associated with a low hepcidin expression.

In a preferred embodiment the subject treated with the EGFR antagonist according to the invention is a male human.

In another embodiment, the chronic liver diseases according to the invention are associated with a low hepcidin expression such as alcoholic liver disease, chronic hepatitis C, or genetic hemochromatosis.

In still another embodiment, the antagonist of the EGF receptor according to the invention bind to EGF receptor, block the binding of EGF on EGFR and block the phosphorylation of the EGFR. To identify an antagonist able to block the interaction between EGF on EGFR, a test based on the effect of the EGFR antagonist candidate on the induction of hepcidin gene expression as explained in the examples (FIG. 5) may be used.

Typically, antagonist according to the invention includes but is not limited to a

i.; erlotinib, gefitinib, canertinib, PD169540, AG1478, PD153035, CGP59326, PKI166; EKB569, or GW572016

ii. an anti-EGFR antibody or antibody fragment that may partially or completely block EGFR activation by EGF

iii. an inhibitor of EGFR, or EGF expression

In another aspect the invention provides an isolated antagonist of the EGF receptor (EGFR), for use in treating chronic liver diseases associated with a low hepcidin expression.

### DEFINITIONS

A “coding sequence” or a sequence “encoding” an expression product, such as an RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, references to specific proteins (e.g., EGFR or EGF) may include a polypeptide having a native amino acid sequence, as well as variants and modified forms regardless of their origin or mode of preparation. A protein that has a native amino acid sequence is a protein having the same amino acid sequence as obtained from nature (e.g., EGFR or EGF). Such native sequence proteins may be isolated from nature or may be prepared using standard recombinant and/or synthetic methods. Native sequence proteins specifically encompass naturally occurring truncated or soluble forms, naturally occurring variant forms (e.g., alternatively spliced forms), naturally occurring allelic variants and forms including postranslational modifications. A native sequence protein includes proteins following post-translational modifications such as glycosylation, or phosphorylation, or other modifications of some amino acid residues.

Variants refer to proteins that are functional equivalents to a native sequence protein that have similar amino acid sequences and retain, to some extent, one or more activities of the native protein. Variants also include fragments that retain activity. Variants also include proteins that are substantially identical (e.g., that have 80, 85, 90, 95, 97, 98, 99%, sequence identity) to a native sequence. Such variants include proteins having amino acid alterations such as deletions, insertions and/or substitutions. A “deletion” refers to the absence of one or more amino acid residues in the related protein. The term “insertion” refers to the addition of one or more amino acids in the related protein. A “substitution” refers to the replacement of one or more amino acid residues by another amino acid residue in the polypeptide. Typically, such alterations are conservative in nature such that the activity of the variant protein is substantially similar to a native sequence protein (see, e.g., Creighton (1984) Proteins, W.H. Freeman and Company). In the case of substitutions, the amino acid replacing another amino acid usually has similar structural and/or chemical properties. Insertions and deletions are typically in the range of 1 to 5 amino acids, although depending upon the location of the insertion, more amino acids may be inserted or removed. The variations may be made using methods known in the art such as site-directed mutagenesis (Carter, et al. (1986) Nucl. Acids Res. 13:4331; Zoller et al. (1987) Nucl. Acids Res. 10:6487), cassette mutagenesis (Wells et al. (1985) Gene 34:315), restriction selection mutagenesis (Wells, et al. (1986) Philos. Trans. R. Soc. London SerA 317:415), and PCR mutagenesis (Sambrook et al., Molecular Cloning: A Laboratory Manual, 3rd edition, Cold Spring Harbor Press, N.Y., (2001)).

Two amino acid sequences are “substantially homologous” or “substantially similar” when greater than 80%, preferably greater than 85%, preferably greater than 90% of the amino acids are identical, or greater than about 90%, preferably grater than 95%, are similar (functionally identical). Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

The term “expression” when used in the context of expression of a gene or nucleic acid refers to the conversion of the information, contained in a gene, into a gene product. A gene product may be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs which are modified, by processes such as capping, polyadenylation, methylation, and editing, and proteins (e.g., EGFR) modified by, for example, methylation, acetylation, phosphorylation, ubiquitination, SUMOylation, ADP-ribosylation, myristilation, and glycosylation.

An ‘inhibitor of expression” refers to a natural or synthetic compound that has a biological effect in inhibiting the expression of a gene.

A “receptor” or “receptor molecule” is a soluble or membrane bound/associated protein or glycoprotein comprising one or more domains to which a ligand binds to form a receptor-ligand complex. By binding the ligand, which may be an agonist or an antagonist the receptor is activated or inactivated and may initiate or block pathway signaling.

By “ligand” or “receptor ligand” is meant a natural or synthetic compound which binds a receptor molecule to form a receptor-ligand complex. The term ligand includes agonists, antagonists, and compounds with partial agonist/antagonist action.

An “agonist” or “receptor agonist” is a natural or synthetic compound which binds the receptor to form a receptor-agonist complex by activating said receptor and receptor-agonist complex, respectively, initiating a pathway signaling and further biological processes.

By “antagonist” or “receptor antagonist” is meant a natural or synthetic compound that has a biological effect opposite to that of an agonist. An antagonist binds the receptor and blocks the action of a receptor agonist by competing with the agonist for receptor. An antagonist is defined by its ability to block the actions of an agonist.

The term “EGFR”, “ErbB” or “HER” refers to a receptor protein tyrosine kinase which belongs, to the ErbB receptor family and includes ErbB1 (or HER1 or EGFR), ErbB2 (or HER2), ErbB3 (or HER 3) and ErbB4 (or HER 4) receptors (Ullrich, 1984). The ErbB receptor will generally comprise an extracellular domain, which may bind an ErbB ligand; a lipophilic transmembrane domain; a conserved intracellular tyrosine kinase domain; and a carboxyl-terminal signaling domain harboring several tyrosine residues which may be phosphorylated. The ErbB receptor may be a native sequence ErbB receptor or an amino acid sequence variant thereof. Preferably the ErbB receptor is native sequence human ErbB receptor. Being activated by their six structurally related agonists-EGF, tumor growth factor α (TGFα), heparin-binding EGF-like growth factor (HB-EGF), amphiregulin, betacellulin and epiregulin—the receptors promote pathways entailing proliferation and transformation. Activated EGFRs homo- or heterodimerize and subsequently autophosphorylation of cytoplasmic tyrosine residues is initiated. These phosphorylated amino acids represent docking sites for a variety of different proteins (Prenzel 2001). Tyrosine phosphorylation of the EGFR leads to the recruitment of diverse signaling proteins, including the Adaptor proteins GRB2 (Growth Factor Receptor-Bound Protein-2) and Nck (Nck Adaptor Protein), PLC-Gamma (Phospholipase-C-Gamma), SHC (Src Homology-2 Domain Containing Transforming Protein), and STATS (Signal Transducer and Activator of Transcription 5).

The expressions “ErbB1” and “HER1” and “EGFR” are used interchangeably herein and refer to human EGFR protein.

The term “EGFR antagonist” or “ErbB antagonist” refers to any ErbB antagonist that is currently known in the art or that will be identified in the future, and includes any chemical entity that, upon administration to a patient, results in inhibition of a biological activity associated with activation of the ErbB in the patient (in particularly the induction of hepcidin gene HAMP as shown in the example), including any of the downstream biological effects otherwise resulting from the binding to ErbB of its natural ligand. Such ErbB antagonist include any agent (chemical entity, anti-EGFR antibody, inhibitor of EGFR expression, . . . ) that may block ErbB activation or any of the downstream biological effects of ErbB activation. Such an antagonist may act by binding directly to the intracellular domain of the receptor and inhibiting its kinase activity. Alternatively, such an antagonist may act by occupying the ligand binding site or a portion thereof of the ErbB receptor, thereby making the receptor inaccessible to its natural ligand so that its normal biological activity is prevented or reduced. Alternatively, such an inhibitor acts by modulating the dimerization of ErbB polypeptides, or interaction of ErbB polypeptide with other proteins. Therefore the term “EGFR antagonist” or “Erb1 antagonist” or “HER1 antagonist” refers to an antagonist of the EGFR protein.

Examples of EGFR antagonists include but are not limited to any of the EGFR antagonists described in Garafalo S. et al. (Exp Opin. Ther Pat 2008) all of which are herein incorporated by reference.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

By “purified” and “isolated” it is meant, when referring to a polypeptide (i.e. interferon) or a nucleotide sequence, that the indicated molecule is present in the substantial absence of other biological macromolecules of the same type. The term “purified” as used herein preferably means at least 75% by weight, more preferably at least 85% by weight, still preferably at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

As used herein, the term “subject” denotes a mammal, such as a rodent, a feline, a canine, and a primate. Preferably a subject according to the invention is a human. Even more preferably a subject according to the invention is a male human

In the context of the present invention, the term “chronic liver diseases” means liver diseases associated with a low hepcidin expression such as alcoholic liver disease, chronic hepatitis C, or genetic hemochromatosis.

Thus, as used herein, “a low hepcidin expression” means an expression level value that is statistically (i.e significantly) lower than the reference value. The reference value may be the expression level as measured in the sample from a healthy human, e.g. blood sample from healthy human when performing, e.g. immunoassay.

For instance; a low hepcidin expression means an expression level of hepcidin decreased of at least 20% compared to the reference value.

### Therapeutic Methods and Uses

The present invention provides for methods and compositions (such as pharmaceutical compositions) for treating liver chronic diseases associated with a low hepcidin expression.

Thus an object of the invention is an EGFR antagonist for use in treating liver chronic diseases associated with a low hepcidin expression.

In one embodiment, the EGFR antagonist is a low molecular weight antagonist.

Low molecular weight EGFR antagonists that may be used in the invention include, for example quinazoline EGFR antagonists, pyrido-pyrimidine EGFR antagonists, pyrimido-pyrimidine EGFR antagonists, pyrrolo-pyrimidine EGFR antagonists, pyrazolo-pyrimidine EGFR antagonists, phenylamino-pyrimidine EGFR antagonists, oxindole EGFR antagonists, indolocarbazole EGFR antagonists, phthalazine EGFR antagonists, isoflavone EGFR antagonists, quinalone EGFR antagonists, and tyrphostin EGFR antagonists, such as those described in the following patent publications, and all pharmaceutically acceptable salts and solvates of said EGFR antagonists: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652.

Additional non-limiting examples of low molecular weight EGFR antagonists include any of the EGFR antagonists described in Traxler, P et al (1998) Exp Opin Ther Patents (UK) 8 and those described in Al-Obeidi F A et al. Oncogene. 2000 Nov. 20; 19(49).

A specific example of a low molecular weight EGFR antagonist that may be used according to the present invention is gefitinib (also known as ZD1839 IRESSA® Astrazeneca) (Woodburn et al., 1997, Proc. Am. Assoc. Cancer Res. 38:633). Iressa is an orally active inhibitor which blocks signal transduction pathways implicated in promoting cancer growth (WO02/28409; WO020020; WO02/005791; WO02/002534; WO01/076586; each of which are incorporated herein by reference). Iressa reportedly has antiangiogenic activity and an antitumor activity against such cancers as colon, breast, ovarian, gastric, non-small lung cancer, pancreatic prostate, and leukemia, it eliminates EGFR, HER2, and HER3 phosphorylation, it inhibits human breast xenograft growth and it has been used in patients (Ciardiello et al. (2001) Clin Cancer Res. 7(5); and Ranson et al. (2002) J Clin Oncol.; 20(9)). Iressa is a quinazoline and has the chemical name 4-quinazolinamine, N-(3-chloro-4-fluorophenyl)-7-methoxy-6-[3-(4-morpholinyl)propoxy]-(9CI) and the chemical formula C22H24ClFN4O3. The Agent is disclosed in International Patent Application WO 96/33980 (Example 1) has the following structure:

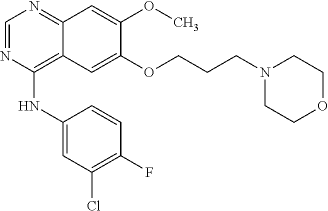 [SMILES: COc1cc2ncnc(Nc3ccc(F)c(Cl)c3)c2cc1OCCCN1CCOCC1]

Another specific example of low molecular weight EGFR antagonist that is used according to the present invention may be the [6,7-bis(2-methoxyethoxy)-4-quinazolin-4-yl]-(3-ethynylphenyl)amine (also known as OSI-774, erlotinib, (erlotinib HCl) Tarceva®) (U.S. Pat. No. 5,747,498; International Patent Publication No. WO 01/34574, and Moyer J D. et al. (1997) Cancer Res. 57(21)). Tarceva has the following structure:

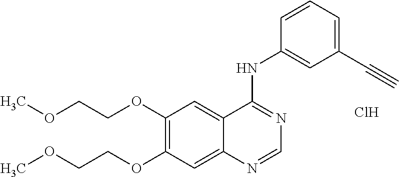 [SMILES: C#Cc1cccc(Nc2ncnc3cc(OCCOC)c(OCCOC)cc23)c1 ; Cl]

Another specific example of a low molecular weight EGFR antagonist is the N-[-4-[(3-Chloro-4-fluorophenyl)amino]-7-[3-(4-morpholinyl)propoxy]-6-quinazolinyl]-2-propenamide Dihydrochloride (known as CI-1033 or PD183805 or Canertinib) (Smaill J B. Et al. (1999) J. Med. Chem., 42; Slichenmyer W J et al. (2001) Semin Oncol. (5 Suppl 16)) and has the following structure:

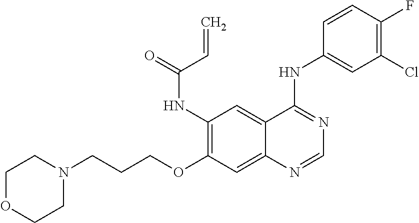 [SMILES: C=CC(=O)Nc1cc2c(Nc3ccc(F)c(Cl)c3)ncnc2cc1OCCCN1CCOCC1]

Another suitable low molecular weight EGFR antagonist is an analog of N-[-4-[(3-Chloro-4-fluorophenyl)amino]-7-[3-(4-morpholinyl)propoxy]-6-quinazolinyl]-2-propenamide Dihydrochloride (CI-1033) known as PD169540 (Smaill J B. Et al. (2000) J Med Chem.; 43(7)).

Another suitable low molecular weight EGFR antagonist is the 4-[(3-bromophenyl)amino]-6-(methylamino)-pyrido[3,4-d]pyrimidine (known as PD-158780) (Rewcastle G W et al. (1998) J Med Chem. 41(5), Cunnick J M et al. (1998) J Biol Chem. 273(23)) and has the following structure:

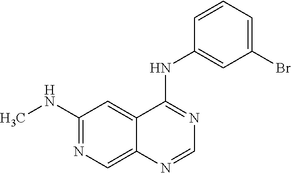 [SMILES: CNc1cc2c(Nc3cccc(Br)c3)ncnc2cn1]

Another suitable low molecular weight EGFR antagonist may be the 4-(3-Chloroanilino)-6,7-dimethoxyquinazoline (known as AG-1478) (University of California)) (Ward W H et al. (1994) Biochem Pharmacol. 48(4); U.S. Pat. No. 5,457,105 and European Patent EP 0,566,266). AG-1478 and has the following structure:

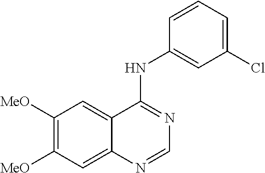 [SMILES: COc1cc2ncnc(Nc3cccc(Cl)c3)c2cc1OC]

Another suitable low molecular weight EGFR antagonist is the 4-[(3-Bromophenyl)amino]-6,7-dimethoxyquinazoline hydrochloride (known as PD 153035) (Bridges A J et al. (1996) J. Med. Chem. 39(1), U.S. Pat. No. 5,457,105 and European Patent 0,566,266) and has the following structure:

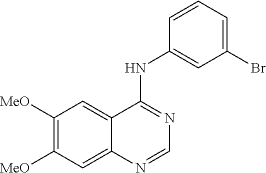 [SMILES: COc1cc2ncnc(Nc3cccc(Br)c3)c2cc1OC]

Another suitable low molecular weight EGFR antagonist is CGP-59326 (Traxler P. et al. (1996) J Med Chem. 39(12)), that has the following structure:

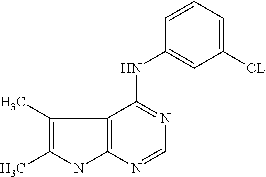

Another suitable low molecular weight EGFR antagonist is the 4-(R)-phenethylamino-6-(hydroxyl)phenyl-7H-pyrrolo[2.3-d]-pyrimidine (known as PKI-166 (Traxler P et al. (1999) Clin. Cancer Res., 5: 3750s) and has the following structure:

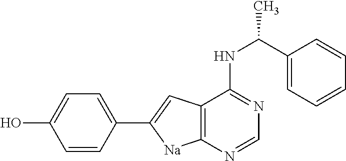

Another suitable low molecular weight EGFR antagonist may be EKB-569 (Torrance C J. et al. (2000)) that has the following structure:

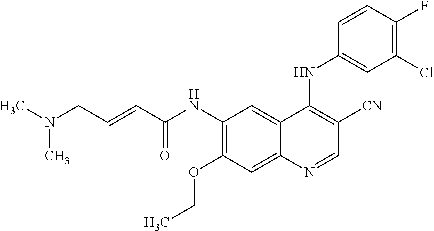 [SMILES: CCOc1cc2ncc(C#N)c(Nc3ccc(F)c(Cl)c3)c2cc1NC(=O)/C=C/CN(C)C]

Another suitable low molecular weight EGFR antagonist may be GW-2016 (also known as GW-572016 or lapatinib ditosylate;) (Kim T E et al. (2003) IDrugs. 6(9):) that has the following structure:

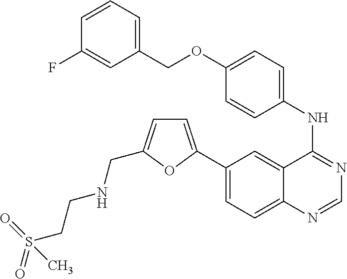 [SMILES: CS(=O)(=O)CCNCc1ccc(-c2ccc3ncnc(Nc4ccc(OCc5cccc(F)c5)cc4)c3c2)o1]

In another embodiment the EGFR antagonist consists in an antibody or antibody fragment that may partially or completely block EGFR activation by EGF.

Non-limiting examples of antibody-based EGFR antagonists include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR antagonist can be the monoclonal antibody Mab E7.6.3 (Yang, X. D. et al. (1999) Cancer Res. 59(6)), or Mab C225 (ATCC Accession No. HB-8508, U.S. Pat. No. 4,943,533), or an antibody or antibody fragment having the binding specificity thereof. Suitable monoclonal antibody EGFR antagonists include, but are not limited to, IMC-C225 (also known as cetuximab), ABX-EGF, EMD 72000, RH3, and MDX-447.

Additional antibody antagonists may be raised according to known methods by administering the appropriate antigen or epitope to a host animal selected, e.g., from pigs, cows, horses, rabbits, goats, sheep, and mice, among others. Various adjuvants known in the art may be used to enhance antibody production. Although antibodies useful in practicing the invention may be polyclonal, monoclonal antibodies are preferred. Monoclonal antibodies against EGFR, or HB-EGF may be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al, 1985, Monoclonal Antibodies and Cancer Therapy, Alan R. Liss, Inc., pp. 77-96). Alternatively, techniques described for the production of single chain antibodies (see, e.g., U.S. Pat. No. 4,946,778) may be adapted to produce anti-EGFR, or anti-EGFR single chain antibodies. EGFR antagonists useful in practicing the present invention also include anti-EGFR, or anti-EGFR antibody fragments including but not limited to F(ab′).sub.2 fragments, which may be generated by pepsin digestion of an intact antibody molecule, and Fab fragments, which may be generated by reducing the disulfide bridges of the F(ab′).sub.2 fragments. Alternatively, Fab and/or scFv expression libraries may be constructed to allow rapid identification of fragments having the desired specificity to EGFR.

Humanized anti-EGFR and antibody fragments therefrom may also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Another object of the invention is an inhibitor of EGFR expression or EGF expression for use in treating liver chronic diseases.

Inhibitors of EGFR or EGF expression for use in the present invention may be based on antisense oligonucleotide constructs. Anti-sense oligonucleotides, including anti-sense RNA molecules and anti-sense DNA molecules, act to directly block the translation of EGFR or HB-EGF mRNA by binding thereto and thus preventing protein translation or increasing mRNA degradation, thus decreasing the level of EGFR or HB-EGF proteins, and thus activity, in a cell. For example, antisense oligonucleotides of at least about 15 bases and complementary to unique regions of the mRNA transcript sequence encoding EGFR or HB-EGF may be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) may also function as inhibitors of EGFR, or EGF expression for use in the present invention. EGFR or EGF gene expression may be reduced by contacting the tumor, subject or cell with a small double stranded RNA (dsRNA), or a vector or construct causing the production of a small double stranded RNA, such that EGFR or EGF expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes may also function as inhibitors of EGFR or EGF expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EGFR or EGF mRNA sequences are thereby useful within the scope of the present invention. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using, e.g., ribonuclease protection assays.

Both antisense oligonucleotides and ribozymes useful as inhibitors of EGFR or EGF expression may be prepared by known methods. These include techniques for chemical synthesis such as, e.g., by solid phase phosphoramadite chemical synthesis. Alternatively, anti-sense RNA molecules may be generated by in vitro or in vivo transcription of DNA sequences encoding the RNA molecule. Such DNA sequences may be incorporated into a wide variety of vectors that incorporate suitable RNA polymerase promoters such as the T7 or SP6 polymerase promoters. Various modifications to the oligonucleotides of the invention may be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing EGFR or EGF. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One may readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N. J., 1991).

Preferred viruses for certain applications are the adeno-viruses and adeno-associated viruses, which are double-stranded DNA viruses that have already been approved for human use in gene therapy. The adeno-associated virus may be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus may integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus may also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, may express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid may be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Another object of the invention relates to a method for treating chronic liver diseases comprising administering a subject in need thereof with a therapeutically effective amount of an antagonist or inhibitor of expression as described above.

In the context of the invention, the term “treating” or “treatment”, as used herein, means reversing, alleviating, inhibiting the progress of, or preventing the disorder or condition to which such term applies, or one or more symptoms of such disorder or condition.

According to the invention, the term “patient” or “patient in need thereof”, is intended for a human or non-human mammal affected or likely to be affected with liver chronic diseases.

By a “therapeutically effective amount” of the antagonist or inhibitor of expression as above described is meant a sufficient amount of the antagonist or inhibitor of expression to treat chronic liver diseases at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Screening Methods

Antagonists of the invention may further be identified by the screening methods described in the state of the art. The screening methods of the invention may be carried out according to known methods.

The screening method may measure the binding of a candidate compound to the EGF receptor, or to cells or membranes bearing the EGF receptor, or a fusion protein thereof by means of a label directly or indirectly associated with the candidate compound. Alternatively, a screening method may involve measuring or, qualitatively or quantitatively, detecting the competition of binding of a candidate compound to the receptor with a labelled competitor (e.g., antagonist or agonist). Further, screening methods may test whether the candidate compound results in a signal generated by an antagonist of the receptor, using detection systems appropriate to cells bearing the EGF receptor. Antagonists may be assayed in the presence of a known agonist (e.g., EGF) and an effect on activation by the agonist by the presence of the candidate compound is observed. Further, screening methods may comprise the steps of mixing a candidate compound with a solution comprising a EGFR, to form a mixture, and measuring the activity in the mixture, and comparing to a control mixture which contains no candidate compound. Competitive binding using known agonist such EGF is also suitable.

### Pharmaceutical Compositions

The antagonist or inhibitor of expression of the invention may be combined with pharmaceutically acceptable excipients, and optionally sustained-release matrices, such as biodegradable polymers, to form therapeutic compositions for use in treating chronic liver diseases associated with a low hepcidin expression.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, may be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The antagonist or inhibitor of expression of the invention may be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, histidine, procaine and the like.

The carrier may also be a solvent or dispersion medium containing, for example, water, ethanol, polyol (for example, glycerol, propylene glycol, and liquid polyethylene glycol, and the like), suitable mixtures thereof, and vegetables oils. The proper fluidity may be maintained, for example, by the use of a coating, such as lecithin, by the maintenance of the required particle size in the case of dispersion and by the use of surfactants. The prevention of the action of microorganisms may be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions may be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions are administered in a manner compatible with the dosage formulation and in such amount as is therapeutically effective. The formulations are easily administered in a variety of dosage forms, such as the type of injectable solutions described above, but drug release capsules and the like may also be employed.

For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which may be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The antagonist or inhibitor of expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses may also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In another embodiment, the pharmaceutical composition of the invention is used in combination with at least one other active ingredient for in treating chronic liver diseases associated with a low hepcidin expression. Example of the other active ingredients is hepcidin (see WO02098444) or synthetic hepcidin (like mini-hepcidin) (see WO2010065815) all of which are herein incorporated by reference.

The invention will further be illustrated in view of the following figures and examples.

### FIGURES

FIG. 1 Bmp6−/− males accumulate more liver iron with age than females and have consistently lower hepcidin mRNA expression than females. Groups of 6 wild-type and 6 Bmp6−/− mice of each gender were compared at 7, 12, and 30 weeks of age. (A) Liver non-heme iron content (mean±SEM) is reported as micrograms of iron per gram dry weight of tissue. At 30 weeks, males have a higher liver iron content than females (10937±1277 vs. 6555±630; p=0.01). (B) Hepcidin (Hamp) mRNA levels were measured by qRT-PCR. Values shown are means of −ΔCt (i.e., Ct Hprt−Ct Hamp)±SEM. The higher the −ΔCt, the greater is the amount of Hamp amplicon. At 7 weeks, hepcidin expression is repressed on average 83.9-fold (−ΔΔCt=1.43-7.82=−6.39; 2−ΔΔCt=1/83.9) in males and only 5.3-fold (−ΔΔCt=5.90-8.32=−2.42; 2−ΔΔCt=1/5.3) in females. Means of −ΔCt values in Bmp6−/− males and females of each age were compared by Student's t-tests and were all significantly different from each other (p=0.001 at 7 weeks, p<0.0001 at 12 weeks, and p=0.002 at 30 weeks).

FIG. 2. Hepcidin mRNA levels in Bmp6^{-/-} males increase after castration. Groups of 12 mice of each sex and age combination were either gonadectomized (N=6) or sham operated (N=6). Hepcidin (Hamp) mRNA levels were measured by qRT-PCR. Values shown are means of −ΔCt (i.e., Ct Hprt−Ct Hamp)±SEM in females (A) and males (B). Means of −ΔCt values in gonadectomized and intact mice of each sex and age combination were compared by Student's t-tests (***, p<0.0001;**, p<0.01).

FIG. 3. Testosterone administration to ovariectomized Bmp6−/− mice represses hepcidin expression. (A) Non-irradiated 7 w.o. ovariectomized mice received daily injections of testosterone propionate (10 mg/kg sc; N=6) or vehicule (N=4) for 7 days. Hepcidin (Hamp) mRNA levels were measured by qRT-PCR. Values shown are means of −ΔCt (i.e., Ct Hprt−Ct Hamp)±SEM. Hepcidin expression was repressed on average 15.4-fold (−ΔΔCt=1.27-5.22=−3.95; 2−ΔΔCt=1/15.4) following treatment with testosterone. (B) Whole-body irradiated mice received daily injections of testosterone (N=8) or vehicule (N=5) from days 2 to 8. Hepcidin expression was reduced on average 16.3-fold (−ΔΔCt=0.81−4.85=−4.03; 2−ΔΔCt=1/16.3) in mice that received testosterone. Means of −ΔCt values in testosterone or vehicule-treated mice were compared by Student's t-tests (***, p<0.001).

FIG. 4. Hepatic expression of epidermal growth factor receptor (Egfr) is testosterone-dependent and activation of Egfr signaling by testosterone coincides with low levels of Smad5 and high levels of Smad2 C-terminal phosphorylation. (A) Egfr mRNA levels were measured by qRT-PCR in 5 males and 5 females Bmp6+/+ as well as 6 males and 6 females Bmp6−/−. Values shown are means of −ΔCt (i.e., Ct Hprt−Ct Egfr)±SEM. Egfr expression was on average 2.9-fold (−ΔΔCt=1.32+0.20=1.52; 2−ΔΔCt=2.87) and 3.2-fold (−ΔΔCt=1.06+0.61=1.67; 2−ΔΔCt=3.18) higher in wild-type and in Bmp6−/− males, respectively, than in the corresponding females. Means of −ΔCt values in gonadectomized and intact mice were compared by Student's t-tests (***, p<0.001). (B) Egfr mRNA levels were measured in 6 gonadectomized and 6 sham-operated 12 w.o. Bmp6−/− males. Egfr expression was reduced on average 3.3-fold (−ΔΔCt=−0.72-1.02=−1.74; 2^{-ΔΔCt}=1/3.34) in castrated males. Egfr mRNA levels were also measured in gonadectomized females treated with vehicule (N=6) or testosterone (N=11) for a week. Egfr expression was increased on average 3.3-fold (−ΔΔCt=0.88+0.84=1.72; 2^{-ΔΔCt}=3.29) following testosterone administration. Means of −ΔCt values in gonadectomized and intact mice or in testosterone and vehicle-treated mice were compared by Student's t-tests (***, p<0.001). (C) Membrane protein extracts were prepared from the mouse livers of Bmp6−/− males and females, castrated males, and gonadectomized females treated with testosterone (4 mice/group). Phospho-Egfr, total Egfr, and vinculin were detected by immunoblot techniques. (D&E) Total protein extracts were prepared from the livers of the same mice and immunoblot techniques were used to detect (D) C-terminal phospho-Smad5 and total Smad5 or (E) C-terminal phospho-Smad2 and total Smad2. Results for two representative mice/group are shown on the blots

FIG. 5. Selective inhibition of Egfr in mice prevents hepcidin downregulation by testosterone. 7 w.o. Bmp6^{-/-} males were treated with the selective EGFR-tyrosine kinase inhibitor, gefitinib, or vehicule daily for 7 days. (A) Fresh membrane protein extracts were prepared from mouse livers. Phospho-Egfr and vinculin were detected by immunoblot techniques. (B) Hamp mRNA levels were measured by qRT-PCR in Bmp6^{-/-} males treated with gefitinib or vehicule. Hamp expression was on average 6.8-fold (−ΔΔCt=4.02-1.26=2.76; 2^{-ΔΔCt}=6.8) higher in mice who received gefitinib than in mice treated with vehicule. Means of −ΔCt values in mice treated with or without gefitinib were compared by Student's t-tests (**, p<0.01).

### EXAMPLE

### Material & Methods

Animals and Treatments.

Bmp6 null mice (Bmp^{6mlRob}) obtained from E. Robertson and wild-type controls on a CD1 background were sacrificed at 7, 12, or 30 weeks of age. Liver, spleen, heart, pancreas and kidney samples were dissected for RNA isolation, flash frozen in liquid nitrogen and stored at −80° C. Hamp-deficient mice were kindly provided by S. Vaulont. They were derived on a C57BL/6 background in the lab of T. Ganz. Gonadectomies and sham operations were performed under anesthesia at 4 weeks of age. Testosterone (10 mg/kg; Sigma) was suspended in corn oil and a total volume of 60 μL per mouse was injected sc everyday for a week. To investigate the role of erythropoiesis in the down-regulation of hepcidin by testosterone, Bmp6−/− females were whole-body irradiated with a sublethal dose of 60Co (6 Gy) on day 1, administered daily doses of testosterone (1 μg/g) starting on day 2, and sacrificed on day 8. To investigate the effect of EGF signaling on testosterone-induced hepcidin down-regulation, the selective EGFR inhibitor Gefitinib (Iressa) was stirred into 1% Tween 80 and administered orally daily from days −1 to 7 to Bmp6−/− males (200 mg/kg; Euromedex). Mice were housed under controlled lighting and temperature conditions, fed a chow of normal iron content (250 mg iron/kg; SAFE, Augy, France) ad libidum, and were fasted for 14 h before they were killed. Experimental protocols were approved by the Midi-Pyrenees Animal Ethics Committee.

Tissue Iron Staining and Quantitative Iron Measurement.

Liver, spleen, heart, pancreas and kidney samples were fixed in 10% buffered formalin and embedded in paraffin. Deparaffinized tissue sections were stained with the Perls Prussian blue stain for non-heme iron and counterstained with nuclear fast red. Quantitative measurement of non-heme iron in the liver was performed as described previously15. Results are reported as micrograms of iron per gram dry weight.

Quantitation of mRNA Levels.

Total RNA from mouse liver was extracted using Trizol (Invitrogen). cDNA was synthesized using MMLV-RT (Promega). The sequences of the primers for target genes and the reference gene Hprt are listed in supplemental Table 1. Quantitative PCR reactions were prepared with LightCycler 480 DNA SYBR Green I Master reaction mix (Roche Diagnostics, Mannheim, Germany) and run in duplicate on a LightCycler 480 Instrument (Roche Diagnostics).

Protein Extraction.

Livers were homogeneized in a FastPrep®-24 Instrument (MP Biomedicals) for 15 sec at 4 m/s. The lysis buffer (50 mM Tris-HCl, pH 8, 150 mM NaCl, 5 mM EDTA, pH 8, 0.1% NP-40) included inhibitors of proteases (complete protease inhibitor cocktail, Roche Applied Science) and of phosphatases (phosphatase inhibitor cocktail 2, Sigma-Aldrich, Saint-Quentin Fallavier, France). Liver proteins were quantified using a protein assay kit (Bio-Rad).

Western Blot Analysis.

Fresh protein extracts were diluted in Laemmli buffer (Sigma-Aldrich), incubated for 5 minutes at 95° C., and subjected to SDS-PAGE. Proteins were then transferred to nitrocellulose membranes (Amersham). Membranes were blocked with 5% of dry milk in TBS-T buffer (10 mM Tris-HCl, pH 7.5, 150 mM NaCl, 0.15% Tween 20), incubated with rabbit Abs to phospho-Smad5 (Epitomics; 1/20 000), phospho-Smad2 (Ser467) (Abcam; 1/1 000), phospho-Smad1 (Ser206) (Cell Signaling; 1/1 000), or phospho-EGFR (Epitomics; 1/5 000) at 4° C. overnight, and washed with TBS-T buffer. After incubation with a goat anti-rabbit IgG Ab (Cell Signaling Technology) conjugated to HRP, enzyme activity was visualized by an ECL-based detection system (Amersham). Blots were then stripped and reprobed with rabbit Abs to Smad5 (Epitomics; 1/20 000) or EGFR (Cell Signaling; 1/5 000), or with the monoclonal anti-Smad2 (Cell Signaling; 1/2 000) or anti-vinculin Abs (Sigma; 1/30 000) for 2 hours at room temperature before incubation with goat anti-rabbit or horse anti-mouse HRP-linked Abs (Cell Signaling; 1/5 000).

Statistical Analyses.

Data were first normalized to the invariant control Hprt and, for each sample and each target gene, −ΔCt=−[Ct target gene−Ct Hprt] was calculated. Because the numerical value of Ct is inversely related to the amount of amplicon in the reaction, the higher the −ΔCt value, the greater the amount of target amplicon. Values shown are means±SEM. Target gene expression in an individual is proportional to 2^{-ΔCt}. However, individual expression values are usually shown on a log scale and, because log 2 (2^{-ΔCt})−ΔCt, −ΔCt data rather than 2^{-ΔCt} data are plotted on the y-axes. An increase of 1 on the y-axis thus corresponds to a 2-fold increase in target gene expression. ΔCt data are the observed values from experimental procedures and it is recommended that ΔCt data rather 2−ΔCt data be the subject of statistical analysis (41). Means of ΔCt values in males and females, or gonadectomised and intact mice, or testosterone challenged or unchallenged mice, were thus compared by Student t tests. All target and Hprt genes had PCR amplification efficiencies close to 2, and therefore point estimates of expression ratios between condition 2 and reference condition 1 were derived from 2−^{ΔΔCt} where −ΔΔCt=−ΔCt condition 2−(−ΔCt reference condition 1).

Results

Bmp6-Deficiency Leads to a Much Stronger Hepcidin Down-Regulation in Males than in Females.

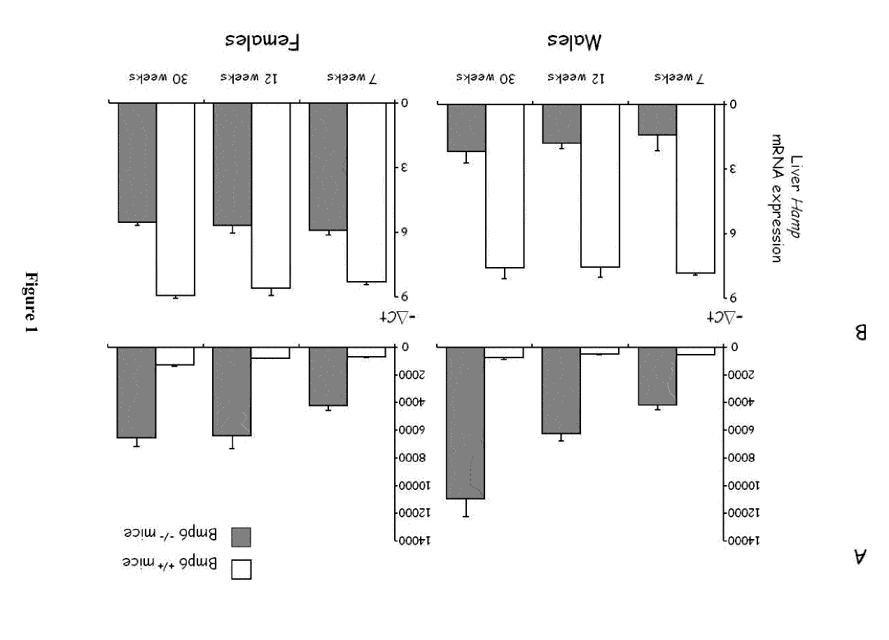

Bmp6 plays a critical role in the maintenance of iron homeostasis. Indeed, 7 w.o. Bmp6^{-/-} mice present with marked iron accumulation in liver parenchymal cells, reduced hepcidin expression compared with wild-type mice, and stabilization of ferroportin at the membrane of enterocytes and tissue macrophages (10). However, although 7 w.o. Bmp6^{-/-} males have about the same amount of liver iron as females (4179±356 vs. 4202±374 μg iron/g dry weight; FIG. 1A), they have a much stronger down-regulation of hepcidin mRNA, compared with wild-type controls (on average 83.9-fold in males and only 5.3-fold in females; FIG. 1B). This prompted us to examine the gender differences in hepcidin regulation further. We quantified hepcidin expression and assessed liver iron accumulation in older (12 and 30 w.o.) mice of both genders. Bmp6^{-/-} males have consistently lower hepcidin mRNA expression than Bmp6^{-/-} females (FIG. 1B). As a consequence, they accumulate more liver iron with age than females (10937±1277 vs. 6555±630 μg iron/g dry weight at 30 weeks; FIG. 1A). The gender-related differences in hepcidin levels previously reported in wild-type mice (15,16) and confirmed in this study are thus magnified in Bmp6^{-/-} mice.

Male but not Female 12 w.o Bmp6−/− Mice Accumulate Iron in the Pancreas, the Heart and the Kidneys.

We next assessed the sites of iron accumulation in males and females by staining histological sections for iron. Interestingly, whereas iron deposition appears restricted to the liver in 12 w.o. females, males of the same age have major iron loading in other tissues, most notably the exocrine pancreas, the heart, and the proximal and distal convoluted tubules of the kidney. These gender differences in tissue iron deposition are exacerbated with age and particularly striking in 30 w.o. mice.

Castration of Bmp6−/− Males Increases Hepcidin Expression and Strongly Reduces Tissue Iron Deposition.

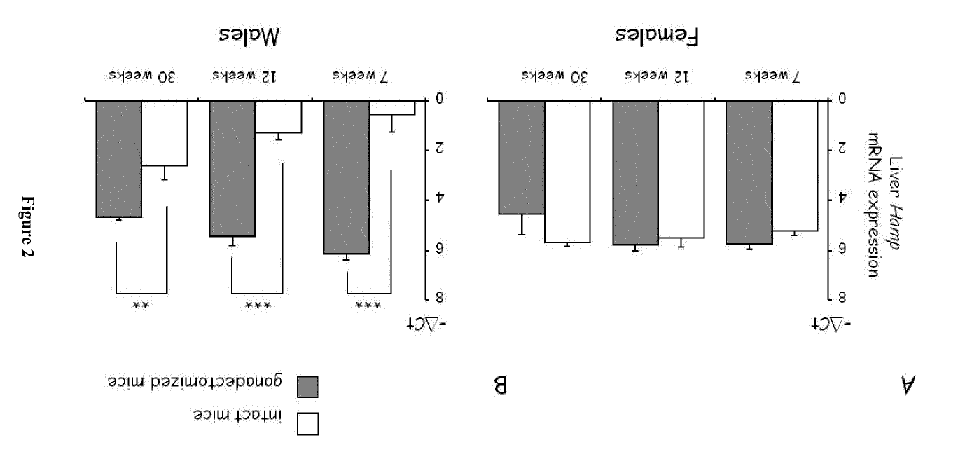

To investigate the reasons for these important gender differences, 4 w.o. Bmp6−/− animals were ovariectomized or castrated. Hepcidin expression is similar in ovariectomized and non-ovariectomized Bmp6^{-/-} females (FIG. 2A). Ovariectomized Bmp6^{-/-} females exclusively accumulate iron in their liver (not shown). In contrast, castrated Bmp6^{-/-} males have much higher hepcidin expression than non-castrated animals (FIG. 2B). Their hepcidin levels are similar to those of Bmp6^{-/-} females of the same age, indicating that male gonadal hormones are responsible for the inhibition of hepcidin expression. The hepatic iron content of 30 w.o. castrated males is equivalent to that of females (6081±241 vs. 5960±107 μg iron/g dry weight). Most remarkably, 12 w.o. castrated Bmp6^{-/-} males have virtually no iron in organs other than the liver and 30 w.o. castrated males have considerably lower iron accumulation in their pancreas and heart than non-castrated males.

Testosterone is the Major Hormone Responsible for the Observed Gender Differences in the Regulation of Iron Metabolism.

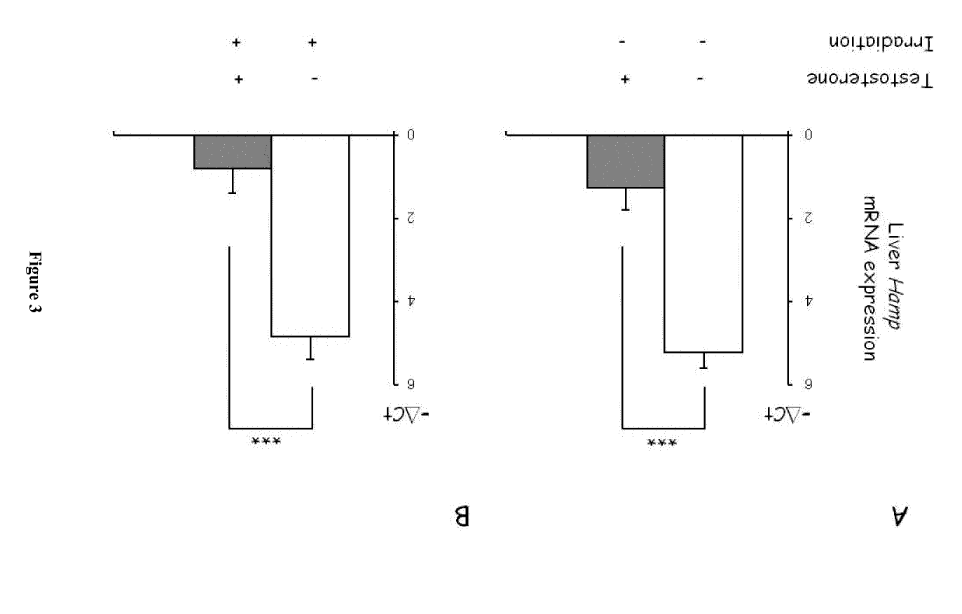

To examine the role of testosterone on hepcidin production further, 7 w.o. ovariectomized Bmp6^{-/-} females received daily injections of testosterone propionate (10 mg/kg sc) or vehicule for a week. As shown on FIG. 3A, hepcidin mRNA expression was repressed on average 15.4-fold after testosterone treatment. Hepcidin expression was reduced in the same proportions (on average 15.8-fold) in 7 w.o. Bmp6^{-/-} males compared with females (FIG. 1B), suggesting that testosterone is the major hormone responsible for the inhibition of hepcidin in males.

Residual Hepcidin Levels in Bmp6^{-/-} Females are Sufficient to Prevent Massive Tissue Iron Loading.

Differences in tissue iron deposition between males and females could be the consequence of reduced production of hepcidin, increased iron absorption, and higher circulating amounts of non-transferrin-bound iron (NTBI) in males compared with females. Alternatively, these differences could be independent of the levels of hepcidin but due to the influence of male gonadal hormones on the expression of iron transporters into storage tissues. To discriminate between these two possibilities, we compared tissue iron accumulation of 12 w.o. hepcidin (Hamp)-deficient males and females. In contrast to Bmp6^{-/-} females, Hamp^{-/-} females accumulate iron not only in the liver, but also in the pancreas, heart and kidneys. This suggests that the residual hepcidin levels found in Bmp6^{-/-} females are sufficient to protect them against massive iron loading of organs other than the liver. Testosterone effects on iron deposition in storage organs are therefore mediated through testosterone-induced down-regulation of hepcidin expression in males rather than upregulation of iron transporters in storage tissues.

Testosterone-Induced Downregulation of Hepcidin Expression is not Due to its Ability to Stimulate Erythropoiesis.

Transcription of the hepcidin gene is controlled negatively by the rate of erythropoiesis (17). Men and women exhibit differences in haemoglobin concentration and during puberty haemoglobin levels increase only in males. Moreover, haemoglobin levels decline after castration or antitestosterone therapy (18). These observations suggest that androgens play a role in erythropoiesis. We first tested whether testosterone has an influence on Epo transcription in the liver and/or the kidney but did not find a significant difference in Epo mRNA levels between Bmp6−/− males and females (data not shown). In humans, levels of erythropoietin are also similar in men and women and it is assumed that testosterone increases the sensitivity of erythroid progenitors to erythropoietin (19). To test whether the down-regulation of hepcidin expression by testosterone in Bmp6^{-/-} mice was due to the stimulation of erythropoiesis, we irradiated ovariectomized females (⁶⁰Co, 6 Gy) to inhibit erythropoiesis. Testosterone propionate (10 mg/kg) or vehicule was then administered on days 2 to 8. Giemsa stain and flow cytometry analysis of bone marrow at day 8 showed massive depletion of nucleated cells in irradiated mice. The spleens of these mice were atrophic and erythropoiesis was absent, indicating no induction of extramedullary erythropoiesis. Furthermore, in the absence of testosterone, hepcidin expression was not reduced in irradiated mice compared with non-irradiated mice, confirming that erythopoiesis was inhibited (FIG. 3). Interestingly, irradiation did not prevent testosteroneinduced downregulation of hepcidin expression to levels similar to those observed in non irradiated control mice (FIG. 3B). These results demonstrate that the observed effects of testosterone on hepcidin expression are not caused by the negative control of erythropoietic regulators.

Activation of Epidermal Growth Factor Receptor (Egfr) Signaling in the Liver is Testosterone-Dependent and Inhibits Hepcidin Expression.

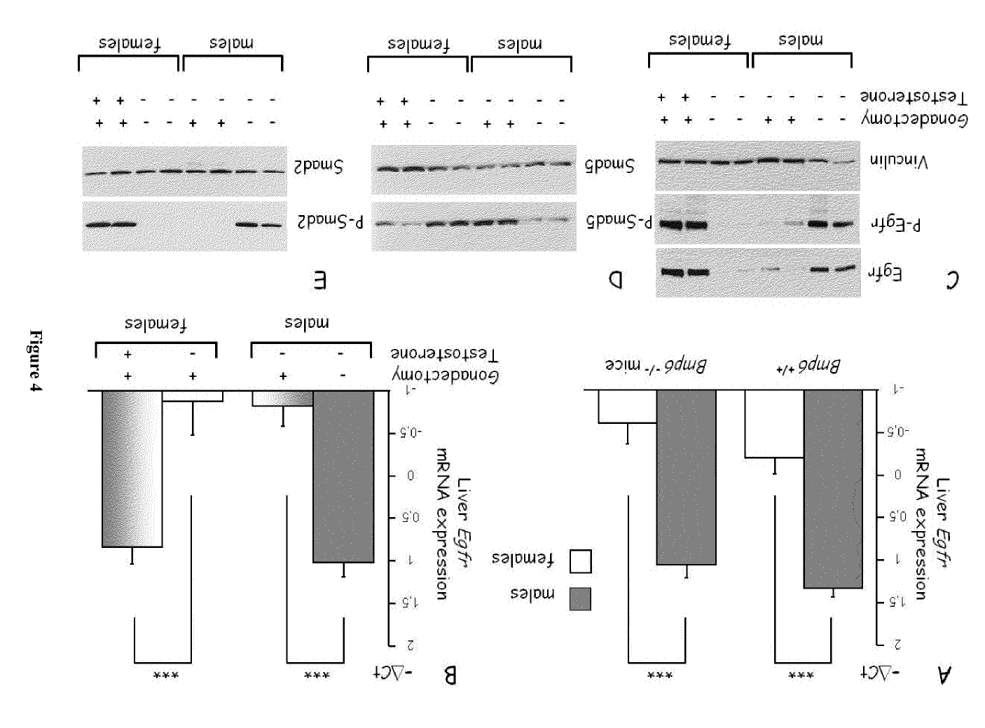

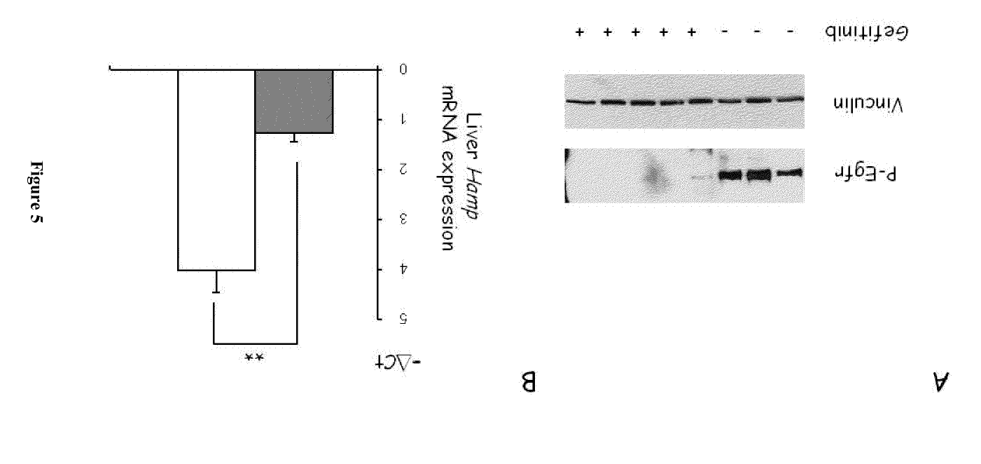

The growth factors EGF and HGF were recently shown to suppress hepatic hepcidin synthesis (20). In vivo, the physiological role of EGF and HGF may depend on target tissue changes in the expression of their receptors, EGFR and Met, which may be modulated by endocrine influences. We therefore compared Egfr and Met mRNA expression between males and females. There was no influence of gender on liver expression of Met (data not shown). In contrast, mRNA expression of Egfr was sexually dimorphic, and higher in males than in females, both in wild-type and Bmp6^{-/-} mice (FIG. 4A). In line with these observations, expression of Egfr was reduced in the liver of castrated Bmp6^{-/-} males, and induced in ovariectomized Bmp6^{-/-} females treated with testosterone for a week (FIG. 4B). Similar data were obtained with wild-type mice. As shown on FIG. 4C, there is a good correspondence between Egfr mRNA expression levels, protein abundance, and the amount of phosphorylated Egf receptors, suggesting a role for testosterone in the activation of the EGFR signaling pathway in the liver. To confirm that the effect of testosterone on hepcidin down-regulation was mediated by an increase in Egfr signaling, we treated 7 w.o. Bmp6^{-/-} males with the selective EGFR-tyrosine kinase inhibitor, gefitinib, or vehicule daily for 7 days. As expected, phosphorylation of the Egf receptors was virtually abolished in the liver of mice treated with gefitinib (FIG. 5A). Interestingly, repression of Egfr signaling by gefitinib effectively led to a significant induction of Hamp mRNA levels (FIG. 5B).

Phosphorylation of Smad5 is Lower in Males than in Females and is Influenced by Testosterone Levels.

We then tested whether testosterone-induced hepcidin repression was due to EGF-mediated perturbation of Smad1/5/8 signaling. MAPK activators such as EGF are known to trigger linker phosphorylation of the Smad proteins and thus prime them for recognition and polyubiquitination by Smurf1, and degradation (21,22). Although this could provide an explanation for the lower hepcidin transcription observed in males, no difference in Smad1 phosphorylation at the linker (inhibitory) site was observed between genders (data not shown). However, males had lower amounts of phosphorylation at the C-terminal (activating) site than females (FIG. 4D). Moreover, C-terminal Smad phosphorylation was increased by castration in males, and reduced by administration of testosterone to females (FIG. 4D), which parallels changes in hepcidin expression. These gender differences in C-terminal Smad5 phosphorylation are not explained by differences in gene expression of any of the Bmp ligands (Bmp2, Bmp4, Bmp5, Bmp6, Bmp7, or Bmp9) between males and females (data not shown). We therefore explored the possibility that, as described recently, small C-terminal domain phosphatases (SCPs) regulate Smad activity in these mice by removing EGF-induced linker phosphorylation (23). SCPs dephosphorylate Smad1 not only at the linker site but also at the C-terminal site24. Our observations therefore fit with dephosphorylation by SCPs. Noticeably SCPs also dephosphorylate Smad2/3 at the linker but not at the C-terminal site. This leads to de-inhibition of the TGF-β pathway (24). As shown on FIG. 4E, C-terminal Smad2 phosphorylation was higher in males than females, and was reduced by castration in males, and increased by administration of testosterone to females. These observations implicate SCPs in mediating the effect of testosterone and EGF on the BMP pathway and on hepcidin expression.

### REFERENCES

Throughout this application, various references describe the state of the art to which this invention pertains. The disclosures of these references are hereby incorporated by reference into the present disclosure.
1. Finberg, K. E. 2011. Unraveling mechanisms regulating systemic iron homeostasis. Hematology Am Soc Hematol Educ Program 2011:532-537.
2. Hentze, M. W., Muckenthaler, M. U., Galy, B., and Camaschella, C. 2010. Two to tango: regulation of Mammalian iron metabolism. Cell 142:24-38.
3. Pigeon, C., Ilyin, G., Courselaud, B., Leroyer, P., Turlin, B., Brissot, P., and Loreal, O. 2001. A new mouse liver-specific gene, encoding a protein homologous to human antimicrobial peptide hepcidin, is overexpressed during iron overload. J Biol Chem 276:7811-7819.
4. Nemeth, E., Tuttle, M. S., Powelson, J., Vaughn, M. B., Donovan, A., Ward, D. M., Ganz, T., and Kaplan, J. 2004. Hepcidin regulates cellular iron efflux by binding to ferroportin and inducing its internalization. Science 306:2090-2093.
5. Nicolas, G., Bennoun, M., Devaux, I., Beaumont, C., Grandchamp, B., Kahn, A., and Vaulont, S. 2001. Lack of hepcidin gene expression and severe tissue iron overload in upstream stimulatory factor 2 (USF2) knockout mice. Proc Natl Acad Sci USA 98:8780-8785.
6. Nicolas, G., Bennoun, M., Porteu, A., Mativet, S., Beaumont, C., Grandchamp, B., Sirito, M., Sawadogo, M., Kahn, A., and Vaulont, S. 2002. Severe iron deficiency anemia in transgenic mice expressing liver hepcidin. Proc Natl Acad Sci USA 99:4596-4601.
7. Roetto, A., Papanikolaou, G., Politou, M., Alberti, F., Girelli, D., Christakis, J., Loukopoulos, D., and Camaschella, C. 2003. Mutant antimicrobial peptide hepcidin is associated with severe juvenile hemochromatosis. Nat Genet 33:21-22.
8. Kautz, L., Meynard, D., Monnier, A., Darnaud, V., Bouvet, R., Wang, R. H., Deng, C., Vaulont, S., Mosser, J., Coppin, H., et al. 2008. Iron regulates phosphorylation of Smad1/5/8 and gene expression of Bmp6, Smad7, Id1, and Atoh8 in the mouse liver. Blood 112:1503-1509.
9. Andriopoulos, B., Jr., Corradini, E., Xia, Y., Faasse, S. A., Chen, S., Grgurevic, L., Knutson, M. D., Pietrangelo, A., Vukicevic, S., Lin, H. Y., et al. 2009. BMP6 is a key endogenous regulator of hepcidin expression and iron metabolism. Nat Genet 41:482-487.
10. Meynard, D., Kautz, L., Darnaud, V., Canonne-Hergaux, F., Coppin, H., and Roth, M. P. 2009. Lack of the bone morphogenetic protein BMP6 induces massive iron overload. Nat Genet 41:478-481.
11. Huang, F. W., Pinkus, J. L., Pinkus, G. S., Fleming, M. D., and Andrews, N. C. 2005. A mouse model of juvenile hemochromatosis. J Clin Invest 115:2187-2191.
12. Niederkofler, V., Salie, R., and Arber, S. 2005. Hemojuvelin is essential for dietary iron sensing, and its mutation leads to severe iron overload. J Clin Invest 115:2180-2186.
13. Krijt, J., Niederkofler, V., Salie, R., Sefc, L., Pelichovska, T., Vokurka, M., and Necas, E. 2007. Effect of phlebotomy on hepcidin expression in hemojuvelin-mutant mice. Blood Cells Mol Dis 39:92-95.
14. Harrison-Findik, D. D. 2010. Gender-related variations in iron metabolism and liver diseases. World J Hepatol 2:302-310.
15. Courselaud, B., Troadec, M. B., Fruchon, S., Ilyin, G., Borot, N., Leroyer, P., Coppin, H., Brissot, P., Roth, M. P., and Loreal, O. 2004. Strain and gender modulate hepatic hepcidin 1 and 2 mRNA expression in mice. Blood Cells Mol Dis 32:283-289.
16. Krijt, J., Cmejla, R., Sykora, V., Vokurka, M., Vyoral, D., and Necas, E. 2004. Different expression pattern of hepcidin genes in the liver and pancreas of C57BL/6N and DBA/2N mice. J Hepatol 40:891-896.
17. Krijt, J., Jonasova, A., Neuwirtova, R., and Necas, E. 2010. Effect of erythropoietin on hepcidin expression in hemojuvelin-mutant mice. Blood Cells Mol Dis 44:257-261.
18. Gordon, A. S., Mirand, E. A., Wenig, J., Katz, R., and Zanjani, E. D. 1968. Androgen actions on erythropoiesis. Ann N Y Acad Sci 149:318-335.
19. Teruel, J. L., Cano, T., Marcen, R., Villafruela, J. J., Rivera, M., Fernandez-Juarez, G., and Ortuno, J. 1997. Decrease in the haemoglobin level in haemodialysis patients undergoing antiandrogen therapy. Nephrol Dial Transplant 12:1262-1263.
20. Goodnough, J. B., Ramos, E., Nemeth, E., and Ganz, T. 2012 Inhibition of hepcidin transcription by growth factors. Hepatology 56:291-299.
21. Kretzschmar, M., Doody, J., and Massague, J. 1997. Opposing BMP and EGF signalling pathways converge on the TGF-beta family mediator Smad1. Nature 389:618-622.
22. Sapkota, G., Alarcon, C., Spagnoli, F. M., Brivanlou, A. H., and Massague, J. 2007. Balancing BMP signaling through integrated inputs into the Smad1 linker. Mol Cell 25:441-454.
23. Knockaert, M., Sapkota, G., Alarcon, C., Massague, J., and Brivanlou, A. H. 2006. Unique players in the BMP pathway: small C-terminal domain phosphatises dephosphorylate Smad1 to attenuate BMP signaling. Proc Natl Acad Sci USA 103:11940-11945.
24. Sapkota, G., Knockaert, M., Alarcon, C., Montalvo, E., Brivanlou, A. H., and Massague, J. 2006. Dephosphorylation of the linker regions of Smad1 and Smad2/3 by small C-terminal domain phosphatases has distinct outcomes for bone morphogenetic protein and transforming growth factor-beta pathways. J Biol Chem 281:40412-40419.
25. Noguchi, S., Ohba, Y., and Oka, T. 1991. Pretranslational enhancement of epidermal growth factor receptor by direct effect of testosterone in mouse liver. Endocrinology 128:2141-2148.
26. Scoccia, B., Kovar, P., and Benveniste, R. 1991. Gonadal and adrenal effects on hepatic epidermal growth factor receptor expression in a murine model. Endocrinology 129:3240-3246.
27. Hadziahmetovic, M., Song, Y., Wolkow, N., Iacovelli, J., Kautz, L., Roth, M. P., and Dunaief, J. L. 2011. Bmp6 regulates retinal iron homeostasis and has altered expression in age-related macular degeneration. Am J Pathol 179:335-348.
28. Jenkitkasemwong, S., Wang, C. Y., Mackenzie, B., and Knutson, M. D. 2012. Physiologic implications of metal-ion transport by ZIP14 and ZIPS. Biometals 25:643-655.
29. Shahani, S., Braga-Basaria, M., Maggio, M., and Basaria, S. 2009. Androgens and erythropoiesis: past and present. J Endocrinol Invest 32:704-716.
30. Calof, O. M., Singh, A. B., Lee, M. L., Kenny, A. M., Urban, R. J., Tenover, J. L., and Bhasin, S. 2005. Adverse events associated with testosterone replacement in middle20 aged and older men: a meta-analysis of randomized, placebo-controlled trials. J Gerontol A Biol Sci Med Sci 60:1451-1457.
31. Coviello, A. D., Kaplan, B., Lakshman, K. M., Chen, T., Singh, A. B., and Bhasin, S. 2008. Effects of graded doses of testosterone on erythropoiesis in healthy young and older men. J Clin Endocrinol Metab 93:914-919.
32. Kim, S. W., Hwang, J. H., Cheon, J. M., Park, N. S., Park, S. E., Park, S. J., Yun, H. J., Kim, S., and Jo, D. Y. 2005. Direct and indirect effects of androgens on survival of hematopoietic progenitor cells in vitro. J Korean Med Sci 20:409-416.
33. Bachman, E., Feng, R., Travison, T., Li, M., Olbina, G., Ostland, V., Ulloor, J., Zhang, A., Basaria, S., Ganz, T., et al. 2010. Testosterone suppresses hepcidin in men: a potential mechanism for testosterone-induced erythrocytosis. J Clin Endocrinol Metab 95:4743-4747.
34. Escobar-Morreale, H. F. 2012. Iron metabolism and the polycystic ovary syndrome. Trends Endocrinol Metab 23:509-515.
35. Luque-Ramirez, M., Alvarez-Blasco, F., Alpanes, M., and Escobar-Morreale, H. F. 2011. Role of decreased circulating hepcidin concentrations in the iron excess of women with the polycystic ovary syndrome. J Clin Endocrinol Metab 96:846-852.
36. Campostrini, N., Traglia, M., Martinelli, N., Corbella, M., Cocca, M., Manna, D., Castagna, A., Masciullo, C., Silvestri, L., Olivieri, O., et al. 2012. Serum levels of the hepcidin-20 iso form in a large general population: The Val Borbera study. J Proteomics 76 Spec No.:28-35.
37. Papanikolaou, G., Samuels, M. E., Ludwig, E. H., MacDonald, M. L., Franchini, P. L., Dube, M. P., Andres, L., MacFarlane, J., Sakellaropoulos, N., Politou, M., et al. 2004. Mutations in HFE2 cause iron overload in chromosome 1 q-linked juvenile hemochromatosis. Nat Genet 36:77-82.
38. Camaschella, C., Roetto, A., and De Gobbi, M. 2002. Juvenile hemochromatosis. Semin Hematol 39:242-248.
39. Harrison-Findik, D. D., Schafer, D., Klein, E., Timchenko, N. A., Kulaksiz, H., Clemens, D., Fein, E., Andriopoulos, B., Pantopoulos, K., and Gollan, J. 2006. Alcohol metabolism-mediated oxidative stress down-regulates hepcidin transcription and leads to increased duodenal iron transporter expression. J Biol Chem 281:22974-22982.
40. Dupic, F., Fruchon, S., Bensaid, M., Borot, N., Radosavljevic, O., Loreal, O., Brissot, P., Gilfillan, S., Bahram, S., Coppin, H., et al. 2002. Inactivation of the hemochromatosis gene differentially regulates duodenal expression of iron-related mRNAs between mouse strains. Gastroenterology 122:745-751.
41. Yuan, J. S., Reed, A., Chen, F., and Stewart, C. N., Jr. 2006. Statistical analysis of realtime PCR data. BMC Bioinformatics 7:85.

1. A method of treating chronic liver disease associated with a low hepcidin expression in a subject in need thereof, comprising the step of
administering to said patient a therapeutic amount of an antagonist of the EGF receptor (EGFR), wherein said chronic liver disease is genetic hemochromatosis.

2. The method according to claim 1 wherein said subject is a male subject.

3. The method according to claim 1, wherein said antagonist of EGFR is selected from the group consisting of:
i.; erlotinib, gefitinib, canertinib, PD169540, AG1478, PD153035, CGP59326, PKI166; EKB569, GW572016 and an anti-EGFR antibody or antibody fragment that partially or completely blocks EGFR activation by EGF.

4. The method of claim 1, wherein said antagonist of EGFR is provided as a pharmaceutical composition comprising said antagonist of EGFR and a pharmaceutically acceptable excipient, diluent or carrier.

5. The method of claim 4, wherein said pharmaceutical composition comprises at least one other active ingredient used in treating chronic liver diseases associated with a low hepcidin expression.

6. The method according to claim 5 wherein the at least one other active ingredient is hepcidine or synthetic hepcidin.
